(19) United States
(12) Patent Application Publication
(10) Pub. No.: US 2026/0174855 A1
(43) Pub. Date: Jun. 25, 2026

(54) DEUTERATED BUPRENORPHINE AS A PROTECTIVE AGENT FOR FETAL SUBJECTS AGAINST FULL-AGONIST OPIOID EXPOSURE

(71) Applicant: BIOVENTURES, LLC, Little Rock, AR (US)

(72) Inventors: Lisa BRENTS, Little Rock, AR (US); Julia TOBACYK, Little Rock, AR (US); Peter CROOKS, Little Rock, AR (US); Venumadhav JANGANATI, Henrico, VA (US)

(21) Appl. No.: 19/103,488

(22) PCT Filed: Aug. 14, 2023
(86) PCT No.: PCT/US2023/072168

§ 371 Date: Feb. 12, 2025

Related U.S. Application Data
(60) Provisional application No. 63/397,750, filed on Aug. 12, 2022.

(51) Int. Cl. A61K 45/06; A61K 9/00; A61K 31/198; A61K 31/485

(52) U.S. Cl. CPC A61K 45/06; A61K 9/0019; A61K 31/198; A61K 31/485

(57) ABSTRACT

Disclosed herein is deuterated buprenorphine as a protective agent for fetal subjects against full-agonist opioid exposure. Use of deuterated buprenorphine prevents or reduces exposure of the fetus to opioids used by the mother. Use of deuterated buprenorphine may prevent or reduces exposure of the fetus to harmful metabolites of buprenorphine.

### CROSS-REFERENCE TO RELATED APPLICATIONS

This application claims priority to U.S. Provisional Application No. 63/397,750 filed on Aug. 12, 2022, the contents of which are incorporated by reference in their entireties.

### STATEMENT REGARDING FEDERALLY SPONSORED RESEARCH

This invention was made with government support under grants 1U54TR001629-01A1, KL2TR000063, and R21DA049585 awarded by the National Institutes of Health. The government has certain rights in the invention.

### BACKGROUND

Embryonic and fetal opioid exposure is a consequence of opioid addiction during pregnancy that has several potential adverse effects. Maternal use, particularly overdose, of highly potent and efficacious opioids, such as fentanyl and its analogues, can cause fetal and maternal death. Chronic exposure to these opioids throughout gestation disrupts neurodevelopment, leading to dependence and withdrawal during the fetal and neonatal periods, respectively, and potentially life-long neurobehavioral alterations that negatively affect quality of life. Currently, there are no drugs approved by the FDA for protecting the fetus from maternal opioid overdose or exposure to opioids. As a result, there is a need for developing compounds that prevent and protect fetus from opioid overdose or exposure by the mother.

### SUMMARY OF THE INVENTION

Disclosed herein is deuterated buprenorphine as a protective agent for fetal subjects. Use of deuterated buprenorphine may prevent or reduce exposure of the fetus to opioids used by the mother. Moreover, use of deuterated buprenorphine may prevent or reduce exposure of the fetus to harmful metabolites of undeuterated buprenorphine. Deuterated buprenorphine reduces the production of a full opioid agonist, norbuprenorphine, a contributor to fetal opioid dependence and neonatal opioid withdrawal syndrome (NOWS), relative to undeuterated buprenorphine. Deuterated buprenorphine protects the fetus from maternal relapse associated with full opioid agonists, including but not limited to fentanyl, compared to undeuterated buprenorphine. Moreover, Deuterated buprenorphine safeguards the mother from opioid-induced toxicity upon relapse, relative to the protection offered by undeuterated buprenorphine. Further in comparison to its non-deuterated counterpart, deuterated buprenorphine exhibits distinct pharmacokinetic and pharmacodynamic characteristics, thereby making deuterated buprenorphine safer for the maternal, fetal, and neonatal subjects relative to the undeuterated version

One aspect of the technology provides for a method comprising administering a deuterated buprenorphine (BUP), or a pharmaceutically acceptable salt thereof, to a subject during the subject's pregnancy or in anticipation of the subject's pregnancy. In some instances, the maternal subject has, or is suspected of having, an opioid addiction and/or dependence. For example, the material subject may have opioid use disorder. In some instances, the material subject is exposed to an opioid during the subject's pregnancy, which in some cases may be a sufficient exposure over one or more instances to place the fetus at risk for fetal opioid dependence and/or neonatal opioid withdrawal syndrome. In some instances the maternal subject suffers from opioid withdrawal or pain. Suitably, the methods described herein may further comprise administering the deuterated buprenorphine to the material subject's neonate.

Another aspect of the technology provides for a method comprising administering a deuterated buprenorphine to a fetal subject. In some instances, the fetal subject is gestating in a maternal subject having, or is suspected of having, an opioid addiction and/or dependence. For example, the material subject may have opioid use disorder. In some instances, the fetal subject is gestating in a material subject exposed to an opioid, which in some cases may be a sufficient exposure over one or more instances to place the fetal subject at risk for fetal opioid dependence and/or neonatal opioid withdrawal syndrome. In some instances, the fetal subject is gestating in a maternal subject suffers from opioid withdrawal or pain. Suitably, the methods described herein may further comprise administering the deuterated buprenorphine to the fetal subject subsequent to birth, which may be referred to as a neonatal subject subsequent to birth. In such a case, the neonatal subject may be directly administered the deuterated buprenorphine.

Also provided for are pharmaceutical compositions for use in the methods described herein. The deuterated buprenorphine may be administered chronically during fetal gestation.

In some embodiments, the deuterated buprenorphine comprises formula

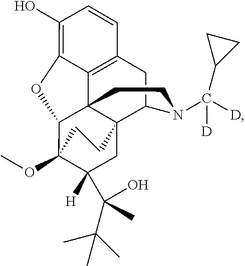

which may be referred to as BUP-D₂ or (4aS,6R,7R,7aR,12bS)-3-(cyclopropylmethyl-d2)-6-((S)-2-hydroxy-3,3-dimethylbutan-2-yl)-7-methoxy-1,2,3,4,5,6,7,7a-octahydro-4a,7-ethano-4,12-methanobenzofuro[3,2-e]isoquinolin-9-ol.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows that BUP-D₂ binds opioid receptors with high affinity. Data points and error bars represent mean and standard error of mean, respectively, of specific receptor binding of the radiolabeled opioid [³H]diprenorphine (y-axis) in homogenates containing hMOR (A), hDOR (B), or hKOR (C) in the presence of varying concentrations (x-axis) of unlabeled morphine (circles), BUP (filled triangles), or BUP-D₂ (unfilled triangles). “Control” refers to [³H]diprenorphine binding in the presence of minimal unlabeled drug (10⁻¹⁴ M). Concentration-dependent inhibition of [³H]diprenorphine binding indicates that the unlabeled drug displaces [³H]diprenorphine from the receptor and therefore has affinity for the receptor. n=3-6 independent experiments.

FIG. 2 illustrates that BUP-D₂ acts as a neutral antagonist of hDORs. [³⁵S]GTPγS specific binding (y-axis) represents G-protein activation in homogenates containing hDOR in the presence of vehicle (0.01% DMSO) or a receptor-saturating concentration (1 μM) of the positive control full agonist DPDPE, the negative control antagonist naltrexone, BUP, or BUP-D₂ (x-axis). Bars represent group means, closed circles represent values obtained from independent experiments, and error bars represent 95% confidence intervals. Columns sharing letters are not significantly different (One-way ANOVA, Tukey's multiple comparison test, p<0.05).

FIG. 3 shows that BUP-D₂ potently activates hMORs and hKORs with low efficacy. [³⁵S]GTPγS specific binding (y-axis) represents G-protein activation in homogenates containing hMOR (A) or hKOR (B) in the presence of increasing concentrations (x-axis) of DAMGO (open circles, A), U50,488 (open circles, B), BUP (closed triangles), or BUP-D₂ (open triangles). Symbols and error bars represent means and standard error of mean from independent experiments (n=4-6).

FIG. 4 shows that BUP-D₂ induces antinociception equal to that of BUP. Data points and error bars represent the mean and standard error, respectively, of the latency for rats to remove their tails from 50° C. water (y-axis) before (baseline, “BL”) and after (10 minutes, A, and 60 minutes, B,) intravenous injection with varying doses (x-axis) of BUP (closed triangles) or BUP-D₂ (open triangles). p>0.05. two-way ANOVA with Sidak's multiple comparisons test, n=3-4.

FIG. 5 illustrates that prenatal exposure to NorBUP induces dependence and withdrawal in neonatal rats. Bars represent group means of neonatal pups' global withdrawal scores; error bars represent S.E.M. Prenatal treatments are plotted on the x-axis and include norbuprenorphine (NorBUP, 0.3-3 mg/kg per day, s.c.), morphine (M, positive control, 15 mg/kg per day, s.c.), and vehicle (V, 1:2:1 DMSO/PEG-400/saline, 0.120 ml/day, s.c.). Neonatal pups were challenged with saline (10 ml/kg, i.p., white bars) or naltrexone (Ntx, 1 mg/kg, i.p., gray bars; Ntx, 10 mg/kg, i.p., black bars) on postnatal day 1 and observed for withdrawal signs for 10 minutes. Ntx precipitates withdrawal signs in opioid-dependent pups. **P, 0.01, ****P, 0.0001 vs. vehicle-treated, saline-challenged pups; Dunnett's multiple comparisons test, n=3-7.

FIG. 6 illustrates that BUP is metabolized by cytochrome P450s (CYPs) to the potentially harmful metabolite NorBUP (A). To decrease metabolism via this pathway, precision deuteration was applied to BUP to make BUP-D₂ (B). The substitution of hydrogen with deuterium interferes with N-dealkylation of the parent drug to NorBUP by CYPs, leading to the distinct pharmacokinetic properties that render BUIP-D2 safer for the fetus compared to its undeuterated form.

FIG. 7 illustrates that less NorBUP, a potentially harmful metabolite of BLIP, is formed from BUP-D2 compared to its non-deuterated form. Velocity of NorBUP formation from the substrates BUP and BUP-D₂ following incubation with human liver microsomes (HLMs A); or SuperSomes™ transfected with CYP19 (B). Data points represent means taken from three independent experiments each performed in triplicate. *p<0.05, **p<0.01, ***p<0.001, main effect of substrate concentration or substrate type by two-way ANOVA, or for BUP vs. BUP-D₂ at each concentration by Sidek's multiple comparisons test.

FIG. 8 shows that metabolic pathways of BUP (A) and BUP-D₂ (B). BUP and BUP-D₂ are subject to glucuronidation to BUP Glucuronide (BUP-Gluc) and BUP-D₂ Glucuronide (BUP-D₂-Gluc), respectively. BUP, BUP-D₂, BUP-Gluc, and BUP-D₂-Gluc are subject to N-dealkylation to form NorBUP (from BUP and BUP-D₂) or NorBUP Glucuronide (NorBUP-Gluc, from BUP-Gluc and BUP-D₂-Gluc). Precision deuteration will interfere with N-dealkylation, shunting metabolism from NorBUP and NorBUP-Gluc to BUP-D₂-Gluc. This will decrease levels of NorBUP and NorBUP-Gluc and increase levels of BUP-D₂ and BUP-D₂-Gluc.

FIG. 9 shows that maximum plasma concentrations (Cₘₐₓ) of BUP-D₂ (A) and BUP-D₂-glucuronide (B) were elevated relative to BUP (A) and BUP-glucuronide (B), respectively, in pregnant rats administered subcutaneous BUP (filled triangles) or BUP-D₂ (open triangles) via osmotic minipumps from GD 9-20. Data points and error bars represent group means and SEM.

FIG. 10 shows that plasma concentrations of the parent drug, BUP or BUP-D₂, (A), the corresponding glucuronide (B), and NorBUP-Gluc (C) were equivalent following administration of a high subcutaneous dose (3 mg/kg/day) of BUP (filled triangles) or BUP-D₂ (open triangles) to pregnant rats from GD 9-20. Data points and error bars represent group means and SEM.

FIG. 11 shows NorBUP plasma concentrations (A) and that NorBUP glucuronide (B) plasma concentrations were lower in rats dosed with BUP-D₂ (4 mg/kg, i.v.) relative to those dosed with BUP (4 mg/kg, i.v.). Data points and error bars represent group means and SEM. Student's two-tailed t-test, p<0.05, n=6.

FIG. 12 shows that saturation of metabolism of BUP and BUP-D₂ occurred in rats following a single high dose (10 mg/kg, i.v.) of BUP or BUP-D₂, potentially precluding observation of group differences by causing a ceiling effect in metabolite plasma concentrations. Increasing dose from 4 mg/kg (bottom two traces) to 10 mg/kg (top two traces) proportionally increased plasma concentrations of BUP and BUP-D₂ (Panel A), but not their metabolites, which is indicated by the overlapping data points on Panels B-D. Data points and error bars represent group means and SEM, n=5-6.

FIG. 13 shows that BUP-D2 mitigates fentanyl-induced catalepsy in pregnant Sprague-Dawley rats. Panels A and B show catalepsy quantification on gestational day (GD) 14 and GD 20, respectively. Experimental groups consisted of pregnant dams equipped with osmotic minipumps administering either a vehicle (n=3), 0.1 mg/kg/day BUP (n=4), or 0.1 mg/kg/day BUP-D2 (n=4) from GD 8-9 till birth. These treatments were concomitantly administered with daily 100 μg/kg fentanyl injections from GD 13 to GD 22. Data points symbolize group means and error bars denote standard errors of the mean (S.E.M).

FIG. 14 shows that BUP-D2 decreases NOWS in the presence of prenatal fentanyl exposure more effectively than BUP. Panels A and B show quantification of neonatal withdrawal signs in females (A) and males (B) following prenatal treatment with vehicle, 0.1 mg/kg/day BUP or 0.1 mg/kg/day BUP-D2. Neonatal pups were challenged with NTX (0, 1 mg/kg, i.p.) to precipitate withdrawals. Points represent group means and error bars represent S.E.M; n=4. *p<0.05 (BUP+FENT vs. BUP-D2+FENT, Šidák's multiple comparisons test.

### DETAILED DESCRIPTION OF THE INVENTION

Disclosed herein is deuterated buprenorphine as a protective agent for fetal subjects against full-agonist opioid exposure. Existing therapeutic strategies focus on treating opioid addiction, most often with the opioid partial agonist buprenorphine (BUP). The disclosed Examples show the use of deuterated buprenorphine prevents or reduces exposure of the fetus to opioids used by the mother. This approach involves administering a deuterated buprenorphine to a subject in an amount that is effective against fetal harm following the subject's use of an opioid.

Opioids may be classified according to their effect at opioid receptors, such as mu opioid receptor (MOR), delta opioid receptor (DOR), or kappa opioid receptor (KOR). Opioids may be full agonists, partial agonists, or antagonists. Agonists interact with a receptor to produce a maximal response from the receptor, such as analgesia following morphine administration. Antagonists bind a receptor but produce no function response while preventing an agonist from binding the receptor. Partial agonists bind a receptor but elicit a partial function response no matter the amount of the partial agonist administered.

Buprenorphine is a high-affinity partial agonist of the mu opioid receptor. It produces less respiratory depression, less dependence and withdrawal, and has lower potential for abuse compared to full agonist opioids like morphine, fentanyl, methadone, heroin, codeine, oxycodone, hydromorphone, or meperidine. Because buprenorphine binds to the same receptors as these opioids, buprenorphine acts as an antagonist against them, thus protecting the fetus from their harmful effects.

Although BUP improves outcomes relative to no treatment or treatment with a full-agonist opioid, BUP treatment during pregnancy is associated with neonatal opioid withdrawal syndrome. Studies have shown that the BUP active metabolite, NorBUP, likely contributes to withdrawal severity in the newborn following prenatal treatment with BUP. In newborns there is a positive correlation between neonatal withdrawal severity and NorBUP concentrations in the umbilical cord plasma (a proxy for fetal exposure) at delivery. Our studies indicate that NorBUP induced NOWS in a rat model of NOWS [1, 2]. In the Examples, use of an in vivo model of neonatal opioid withdrawal syndrome (NOWS) shows that prenatal exposure to NorBUP leads to neonatal withdrawal. In addition to protecting the fetus against exposure to full agonist opioids, deuterated buprenorphine represents an improvement over BUP by decreasing its metabolism to the active metabolite norbuprenorphine (NorBUP).

The annual economic burden of neonatal opioid withdrawal syndrome continues to grow. The benefits of implementing deuterated buprenorphine into clinical care include fewer maternal and fetal complications, subsequently translating to fewer and/or shorter hospital stays and lower hospitalization costs. Deuterated buprenorphine reduces harm to the fetus by acting as a fetal protectant and is a further improvement of BUP due to its reduced metabolism to an active metabolite.

BUP is an opioid of formula:

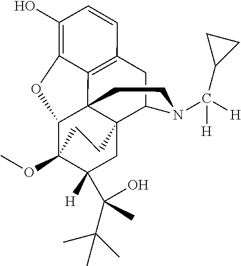

that can be used to treat opioid use disorder, acute pain, or chronic pain. BUP is a high affinity partial agonist of human mu opioid receptor (hMOR) and high affinity antagonist of human delta opioid receptor (hDOR) and human kappa opioid receptor (hKOR). Accordingly, BUP is a nonselective, mixed agonist-antagonist opioid receptor modulator.

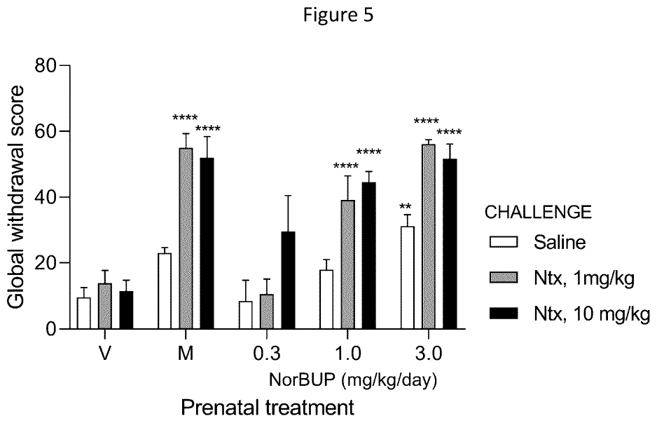

NorBUP, a metabolite of BUP, contributes to NOWS. NorBUP is a high-affinity, high-potency full agonist of MORs [3] that can induce NOWS in a rat model (FIG. 5) [1, 2]. NorBUP concentrations in the human placenta and meconium are at least 10-fold higher than BUP concentrations [4, 5], suggesting that fetal exposure to NorBUP substantially exceeds BUP exposure. Furthermore, NorBUP, but not BUP, concentrations in umbilical cord blood at delivery positively correlate with NOWS severity [6]. Decreasing fetal exposure to NorBUP has the potential to improve short-term neonatal outcomes (e.g. NOWS) as well as long-term effects on fetal and neonatal neurodevelopment.

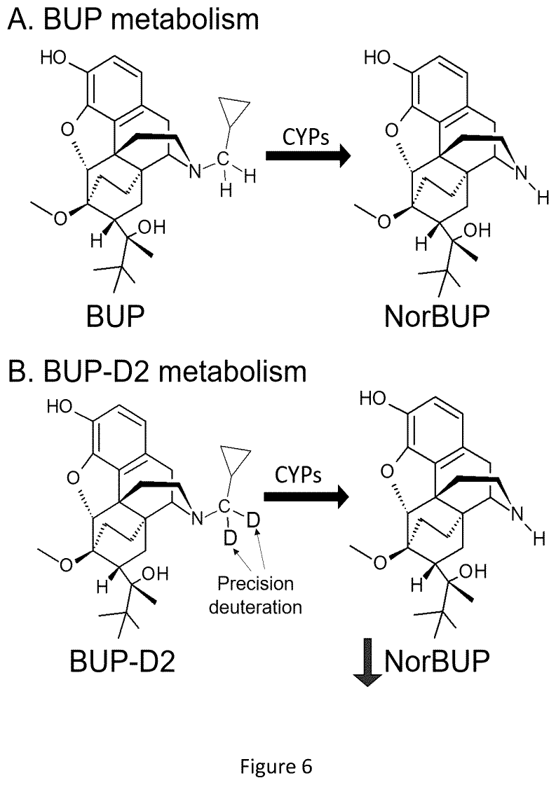

Deuterated buprenorphine is used to decrease NorBUP formation, thereby decreasing fetal exposure. Deuteration of BUP provides for altering pharmacokinetics (such as, metabolism) of BUP by exchanging hydrogen with its heavier isotope deuterium in areas of the molecule that are susceptible to oxidative metabolic cleavage [7, 8]. The exchange strengthens the bonds and makes the N-substituent less susceptible to oxidative cleavage [9]. Here, the Examples show how site-specific deuteration of buprenorphine is accomplished to shunt metabolism from the NorBUP pathway in order to produce less of this active metabolite (FIG. 6). The Examples demonstrate that site-specific deuteration of BUP resists metabolism to NorBUP.

Site specific substitution of atoms having the same atomic number but an atomic mass or mass number different from the atomic mass or mass number that predominates in nature can be regarded as a substituent of a compound of the present disclosure. A sample of a compound having such an isotope as a substituent has at least 50% isotope incorporation at the labelled position(s). The concentration of such isotopes, e.g., deuterium, may be defined by the isotopic enrichment factor. The term “isotopic enrichment factor” as used herein means the ratio between the isotopic abundance and the natural abundance of a specified isotope. For example, if a substituent in a compound of this invention is denoted deuterium, such compound has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

In some embodiments, the methylcyclopropyl group of BUP is specifically substituted with one or more deuterium atoms. In an exemplary embodiment, both hydrogen atoms of the methylene group may be substituted with deuterium atoms. Such a substitution pattern provides for BUP-D₂, a compound having formula.

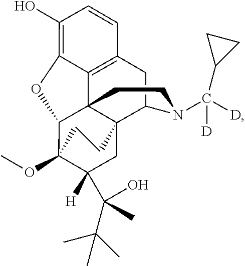

which may be referred to as BUP-D₂ or (4aS,6R,7R,7aR,12bS)-3-(cyclopropylmethyl-d2)-6-((S)-2-hydroxy-3,3-dimethylbutan-2-yl)-7-methoxy-1,2,3,4,5,6,7,7a-octahydro-4a,7-ethano-4,12-methanobenzofuro[3,2-e]isoquinolin-9-ol.

The compounds utilized in the methods disclosed herein may be formulated as pharmaceutical compositions that include: (a) a therapeutically effective amount of one or more deuterated buprenorphine as described herein and (b) one or more pharmaceutically acceptable carriers, excipients, or diluents. The pharmaceutical composition may include the compound in a range of about 0.1 to 2000 mg (preferably about 0.5 to 500 mg, and more preferably about 1 to 100 mg). The pharmaceutical composition may be administered to provide the compound at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the compound at the site of action is about 2 to 10 μM.

The compounds utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof.

The compounds utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The compounds utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents.

Suitable diluents may include pharmaceutically acceptable inert fillers, such as microcrystalline cellulose, lactose, dibasic calcium phosphate, saccharides, and mixtures of any of the foregoing.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Alternatively, only the sodium bicarbonate component of the effervescent couple may be present.

The compounds utilized in the methods disclosed herein may be formulated as a pharmaceutical composition for delivery via any suitable route. For example, the pharmaceutical composition may be administered via oral, intravenous, intramuscular, subcutaneous, topical, transdermal system, subdermal implant, buccal film, and pulmonary route. Examples of pharmaceutical compositions for oral administration include capsules, syrups, concentrates, powders and granules.

The compounds utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the compounds may be adapted for administration by any appropriate route, for example by the oral (including buccal or sublingual), rectal, nasal, topical (including buccal, sublingual, subdermal or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers.

The compounds employed in the compositions and methods disclosed herein may be administered as pharmaceutical compositions and, therefore, pharmaceutical compositions incorporating the compounds are considered to be embodiments of the compositions disclosed herein. Such compositions may take any physical form, which is pharmaceutically acceptable; illustratively, they can be orally administered pharmaceutical compositions. Such pharmaceutical compositions contain an effective amount of a disclosed compound, which effective amount is related to the daily dose of the compound to be administered. Each dosage unit may contain the daily dose of a given compound or each dosage unit may contain a fraction of the daily dose, such as one-half or one-third of the dose. The amount of each compound to be contained in each dosage unit can depend, in part, on the identity of the particular compound chosen for the therapy and other factors, such as the indication for which it is given. The pharmaceutical compositions disclosed herein may be formulated so as to provide quick, sustained, or delayed release of the active ingredient after administration to the patient by employing well known procedures. The compounds for use according to the methods of disclosed herein may be administered as a single compound or a combination of compounds.

Pharmaceutically acceptable salts of the compounds are contemplated and also may be utilized in the disclosed methods. The term “pharmaceutically acceptable salt” as used herein, refers to salts of the compounds that are substantially non-toxic to living organisms. Typical pharmaceutically acceptable salts include those salts prepared by reaction of the compounds as disclosed herein with a pharmaceutically acceptable mineral or organic acid or an organic or inorganic base. Such salts are known as acid addition and base addition salts. It will be appreciated by the skilled reader that most or all of the compounds as disclosed herein are capable of forming salts and that the salt forms of pharmaceuticals are commonly used, often because they are more readily crystallized and purified than are the free acids or bases.

The particular counter-ion forming a part of any salt of a compound disclosed herein is may not be critical to the activity of the compound, so long as the salt as a whole is pharmacologically acceptable and as long as the counter-ion does not contribute undesired qualities to the salt as a whole. Undesired qualities may include undesirably solubility or toxicity.

Pharmaceutically acceptable esters and amides of the compounds can also be employed in the compositions and methods disclosed herein. Examples of suitable esters include alkyl, aryl, and aralkyl esters, such as methyl esters, ethyl esters, propyl esters, dodecyl esters, benzyl esters, and the like. Examples of suitable amides include unsubstituted amides, monosubstituted amides, and disubstituted amides, such as methyl amide, dimethyl amide, methyl ethyl amide, and the like.

In addition, the methods disclosed herein may be practiced using solvate forms of the compounds or salts, esters, and/or amides, thereof. Solvate forms may include ethanol solvates, hydrates, and the like.

An aspect of the technology provides for a method for treating of subject in need of any of compounds described herein. Suitably, method may comprise administering an effective amount of the compound to the subject. As used herein, the terms “treating” or “to treat” each mean to alleviate symptoms, eliminate the causation of resultant symptoms either on a temporary or permanent basis, and/or to prevent or slow the appearance or to reverse the progression or severity of resultant symptoms of the named disease or disorder. As such, the methods disclosed herein encompass both therapeutic and prophylactic administration. As demonstrated in the Examples, the presently disclosed methods prevent or alleviate fetal opioid dependence, NOWS, and other opioid-related toxicities in the fetus, neonate, and mother.

As used herein, a “subject” may be interchangeable with “patient” or “individual” and means an animal, which may be a human or non-human animal, in need of treatment. A “subject in need of treatment” may include a subject having a disease, disorder, or condition that is responsive to therapy with the compounds disclosed herein, either alone or in combination with another bioactive agent.

The subject in need of treatment may be a fetal subject. Accordingly, the compounds described herein may be administered to the fetal subject. Administration to the fetal subject may occur by administration to a maternal subject, in which the fetal subject is gestating. Deuterated buprenorphine can cross the placental barrier, thereby blocking full agonist opioids from binding to the opioid receptors of the fetal subject's brain. Additionally, deuterated buprenorphine reduces prenatal exposure to NorBUP, which can lead to neonatal withdrawal.

The subject in need of treatment may be a maternal subject during the maternal subject's pregnancy or in anticipation of pregnancy. The maternal subject may have, or be suspected of having, opioid addiction, opioid withdrawal, or pain associated with opioid withdrawal. The maternal subject may have, or be suspected of having, exposure to an opioid. Maternal subject exposure to the opioid may be one time, periodic over a period of time, or irregular and repeated over a period of time. As used herein, periodic over a period of time refers to scheduled or planned exposure to an opioid at regularly spaced intervals. As used herein, irregular and repeated over a period of time refers to repeated but unscheduled or unplanned exposure to an opioid at various intervals. In some instances, the maternal subject will be undergoing opioid maintenance therapy. The opioid maintenance therapy may include treatment with a full agonist, such as methadone, partial agonist, such as BUP or BUP-D₂, antagonist, such as naloxone, or any combination thereof, e.g., BUP and naloxone. In some instances, the maternal subject will have had one or more exposures to an opioid that may or will contribute to NOWS in a fetal subject gestating in the maternal subject. Administration to the maternal subject may be useful to treat the maternal subject for opioid addiction, opioid withdrawal, and/or pain while also providing fetal protection to a fetus by blocking full agonist opioids from binding to the opioid receptors of the fetus's brain and reducing prenatal exposure to NorBUP.

In some instances, the maternal subject may suffer from opioid use disorder. Opioid use disorder may be characterized by compulsive use of opioid drugs even when a subject intends to stop use or when using the drugs negatively affects the person's physical and/or emotional well-being. Subjects suffering from opioid use disorder may display symptoms such as physical dependence (e.g., withdrawal symptoms or pain), cravings (e.g., physical or emotional urges to take the opioid), and/or heavy, frequent, unhealthy, or risky use of the opioid. The Diagnostic and Statistical Manual of Mental Disorders, Fifth Edition (DSM-5) may be used to diagnose subjects suffering from opioid use disorder and identify maternal subjects who may benefit from administration of the deuterated BUP during pregnancy or in anticipation of pregnancy.

As used herein, a maternal subject in anticipation of pregnancy includes those subjects planning to become pregnant as well as those who are capable of conceiving, this includes subjects of child-bearing age, health, and/or condition. The methods and compositions used herein may be useful for those who are pregnant, are planning on becoming pregnant, or who may inadvertently become pregnant because the Examples demonstrated that a deuterated BUP is safer for both maternal and fetal subject compared to the undertreated counterpart. In some embodiments, deuterated buprenorphine may be administered to a maternal subject on opioid maintenance therapy that is trying to conceive. In some embodiments, deuterated buprenorphine may be administered to a maternal subject on opioid maintenance therapy that is undergoing infertility treatment. By administering deuterated buprenorphine to maternal subjects in anticipation of pregnancy, those subjects may be administered deuterated buprenorphine throughout their entire gestation period, starting from first to last day of pregnancy.

The compounds described herein may be administered chronically during fetal gestation. As used wherein, chronic administration is the administration of the compounds periodically over an extended period of time. For example, the compounds may be administered for a period of at least 1 week, 4 weeks, 8 weeks, 12 weeks, 16 weeks, 20 weeks, 24 weeks, 28 weeks, 32 weeks, 36 weeks, 40 weeks, or for substantially the known duration of gestation.

As used herein the term “effective amount” refers to the amount or dose of the compound, such as upon single or multiple dose administration to the subject, which provides the desired effect. The effective amount may be an amount that blocks full agonist opioid from binding to the opioid receptors of the fetal subject's brain or that prevents or alleviates the symptoms of NOWS. The effective amount may also be an amount that treats a maternal subject during pregnancy for opioid use disorder or that prevents or alleviates opioid withdrawal. In some instances, the effective amount is selected to treat the fetal and maternal subjects simultaneously, thereby preventing or alleviating the symptoms of NOWS in a fetal subject and treating opioid use disorder or preventing or alleviating opioid withdrawal in a maternal subject.

An effective amount can be determined by the attending diagnostician, as one skilled in the art, by the use of known techniques and by observing results obtained under analogous circumstances. In determining the effective amount or dose of compound administered, a number of factors can be considered by the attending diagnostician, such as: the species of the subject; its size, age, and general health; the degree of involvement or the severity of the disease or disorder involved; the response of the individual subject; the particular compound administered; the mode of administration; the bioavailability characteristics of the preparation administered; the dose regimen selected; the use of concomitant medication; and other relevant circumstances.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

BUP-D₂ Binds Opioid Receptors with High Affinity and Activates them with Low Efficacy

We investigated the pharmacodynamic properties (receptor affinity, potency, and efficacy) of BUP-D₂ for the G protein-coupled opioid receptor subtypes mu, kappa, and delta using in vitro assays of opioid receptor binding and G-protein activation. We also assessed in vivo pharmacodynamics of BUP-D₂ using a rat model of antinociception. We compared these properties of BUP-D₂ to those of non-deuterated BUP, morphine, or other opioids as positive controls.

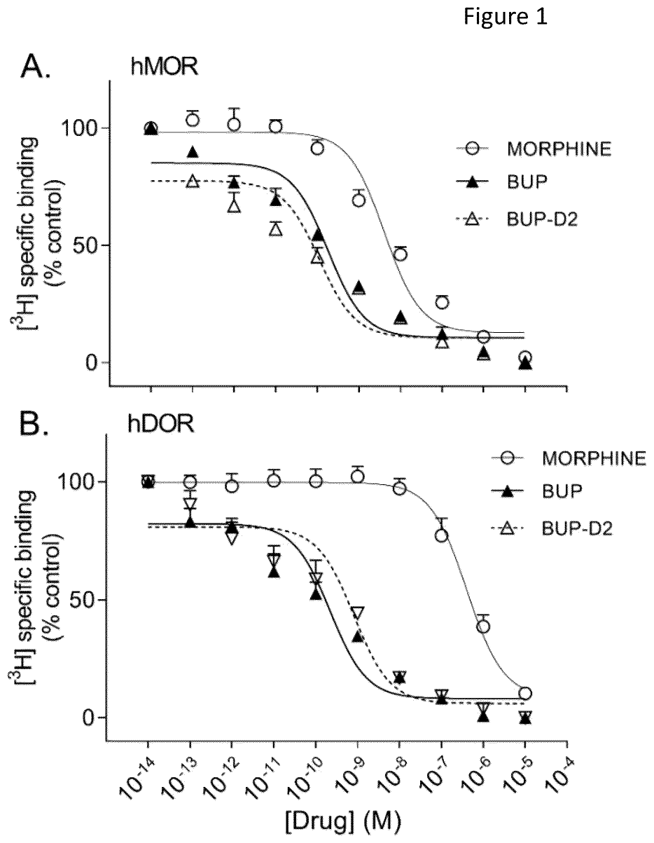

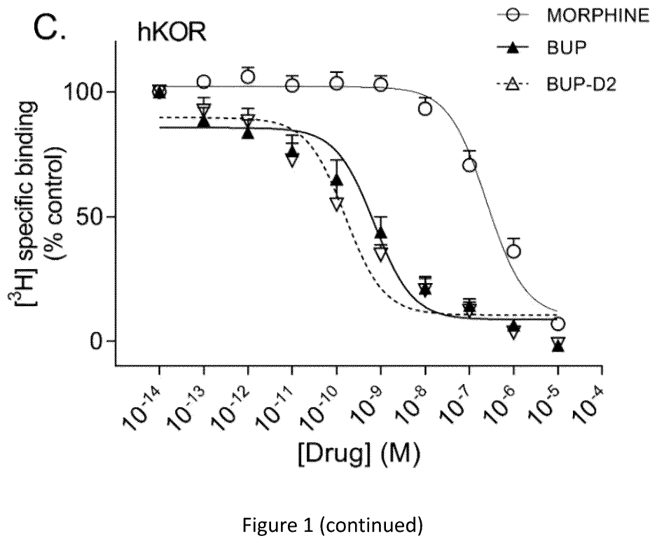

To block the effects of full agonist opioids, such as fentanyl, BUP-D₂ must bind opioid receptors with high affinity. We used a competition receptor binding assay to determine the affinities of BUP-D₂ for human mu opioid receptors (hMOR), human delta opioid receptors (hDOR), and human kappa opioid receptors (hKOR). Morphine and non-deuterated BUP were used as positive controls, and affinity is reported here as a Kᵢ value, which is a measure of affinity describing the concentration of a drug required to bind half of the receptors present in the sample. A smaller Kᵢ value indicates higher affinity (i.e., less drug is required to bind half of the receptors). BUP-D₂ exhibited over 22-fold greater affinity than morphine for hMORs (Kᵢ=0.0532 vs. 1.954 nM, respectively; FIG. 1, Panels A), the opioid receptor subtype that mediates most effects of opioids, including respiratory depression, analgesia, and opioid dependence. BUP-D₂ and BUP exhibited approximately equal affinity for hMORs (Kᵢ=0.0532 vs. 0.0859 nM, respectively; FIG. 1, Panels A). Previous studies have reported that fentanyl binds to hMORs with affinity ranging from 0.39 to 1.9 nM, suggesting that BUP-D₂ has greater affinity than fentanyl for hMORs and that BUP-D₂ can likely block fentanyl from binding to hMORs [10-12].

In addition to hMORs, hDORs and hKORs are bound and activated by many opioids and can therefore contribute to the adverse effects of prenatal full agonist opioid exposure. For this reason, it important that BUP-D₂ bind to hDORs and hKORs in a similar manner as BUP. BUP-D₂ exhibited high affinity for hDORs and hKORs (Kᵢ=0.385 and 0.0749 nM, respectively; FIG. 1, Panels B-C). These were comparable to BUP affinities for hDORs and hKORs (Kᵢ=0.105 and 0.339 nM, respectively; FIG. 1, Panels B-C), and were significantly greater than morphine affinities for hDORs and hKORs (Kᵢ=191.3 and 116.5 nM, respectively; FIG. 1, Panels B-C). Fentanyl has lower affinity than BUP-D₂ for hDOR (Kᵢ ranging from 153-1000 nM) [10, 11] and hKOR (Kᵢ ranging from 85-255 nM) [11]; therefore, as with hMORs, we predict that BUP-D₂ can block fentanyl from binding to hDORs and hKOR.

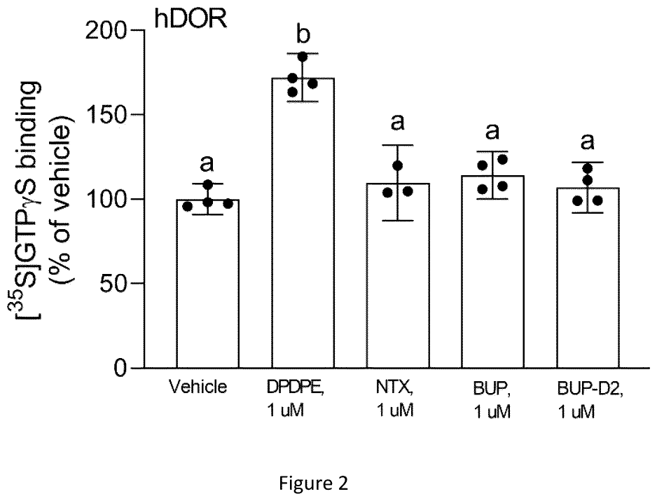

After determining that BUP-D₂ binds opioid receptors with high affinity, we measured the potency and efficacy of BUP-D₂ to activate G-proteins via each opioid receptor subtype. This was important for understanding how BUP-D₂ would affect opioid signaling. To accomplish this goal, we used a non-hydrolyzable, radiolabeled analogue of GTP, called [³⁵S]GTPγS, to bind and label active G-proteins in homogenates containing each opioid receptor type. Activation of G-proteins in this assay increases [³⁵S]GTPγS binding. We measured [³⁵S]GTPγS binding in the presence of vehicle, BUP, BUP-D₂, or a full agonist positive control (DAMGO, DPDPE, and U50,488 for hMOR, hDOR, and hKOR, respectively). Our initial testing with a single, receptor-saturating concentration of each ligand determined that BUP-D₂ and BUP activate hMORs and hKORs with partial agonist activity (data not shown). Neither BUP-D₂ nor BUP activated hDORs (FIG. 2), which is consistent with previously reported findings for BUP [4]. This lack of efficacy coupled with high affinity (FIG. 1, Panel B) suggests BUP-D₂ and BUP are neutral antagonists of hDORs.

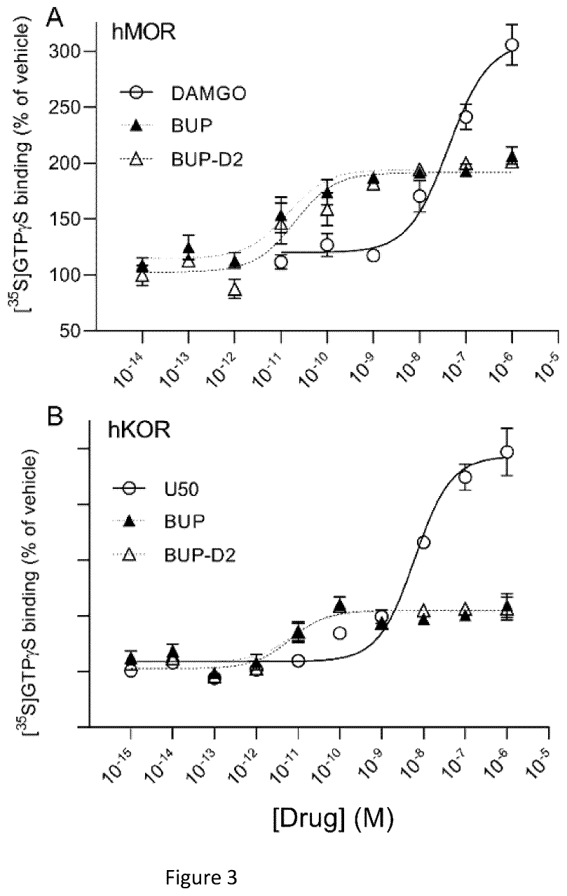

Because the single, receptor-saturating concentration of BUP-D₂ activated hMORs and hKORs, we next measured the concentration-dependence of BUP-D₂ to activate hMOR and hKOR (FIG. 3, Panels A and B). From each concentration-effect curve, we determined an EC₅₀ value, which is the concentration required to produce a half-maximal response. BUP and BUP-D2 exhibited equal potency to activate G-proteins via hMOR and via hKOR (FIG. 3, Panels A and B; Table 1). These concentration-effect curves also indicate that BUP-D₂ and BUP activate hMOR and hKOR with partial agonist activity (i.e., greater than vehicle but less than full agonist DAMGO and U50, 488, respectively). The finding that BUP-D₂ activates hMOR and hKOR with high potency and low efficacy suggests that it can likely prevent maternal opioid withdrawal and cravings while blocking full agonist opioids taken during a relapse.

TABLE 1
Affinity, potency, and efficacy of
BUP-D₂ for opioid receptors, mean (95% CI)

| | | |
|---|---|---|
| hMOR | hDOR | hKOR |

Log Ki values (M)

| | | | |
|---|---|---|---|
| BUP-D2 | −10.3 | −9.54 | −10.21 |
| | (−11.09-−9.51) | (−10.82-−8.26) | (−11.75-−8.66) |
| BUP | −10 | −9.96 | −9.55 |
| | (−10.23-−9.78) | (−10.47-−9.45) | (−11.08-−8.01) |
| Morphine | −8.66 | −6.59 | −6.71 |
| | (−9.07-−8.25) | (−7.11-−6.07) | (−7.60-−5.83) |
| DAMGO | — | — | — |
| DPDPE | — | — | — |
| U50-488 | — | — | — |

EC50 (pM)

| | | | |
|---|---|---|---|
| BUP-D2 | 19.69 | NA | 6.55 |
| | (4.96-180.8) | | (2.14-19.01) |
| BUP | 13.97 | NA | 7.19 |
| | (4.19-72.32) | | (1.93-25.20) |
| Morphine | — | — | — |
| DAMGO | 42100 | — | — |
| | (17720-93440) | | |
| DPDPE | — | — | — |
| U50-488 | — | — | 6155 |
| | | | (2697-12730) |

Emax (% of vehicle)

| | | | |
|---|---|---|---|
| BUP-D2 | 191.5 | *106.94 | 155 |
| | (178.1-208.6) | (92.00-121.90) | (147.5-162.6) |
| BUP | 194.5 | *114.32 | 154.1 |
| | (183.3-206.4) | (100.3-128.33) | (146.4-161.9) |
| Morphine | — | — | — |
| DAMGO | 307.8 | — | — |
| | (280.5-339.4) | | |
| DPDPE | — | *172.00 | — |
| | | (157.75-186.19) | |
| U50-488 | — | — | 293.1 |
| | | | (275.2-311.8) |

NA, not applicable;
“—”, not determined;
*determined from single-concentration (1 μM) experiments

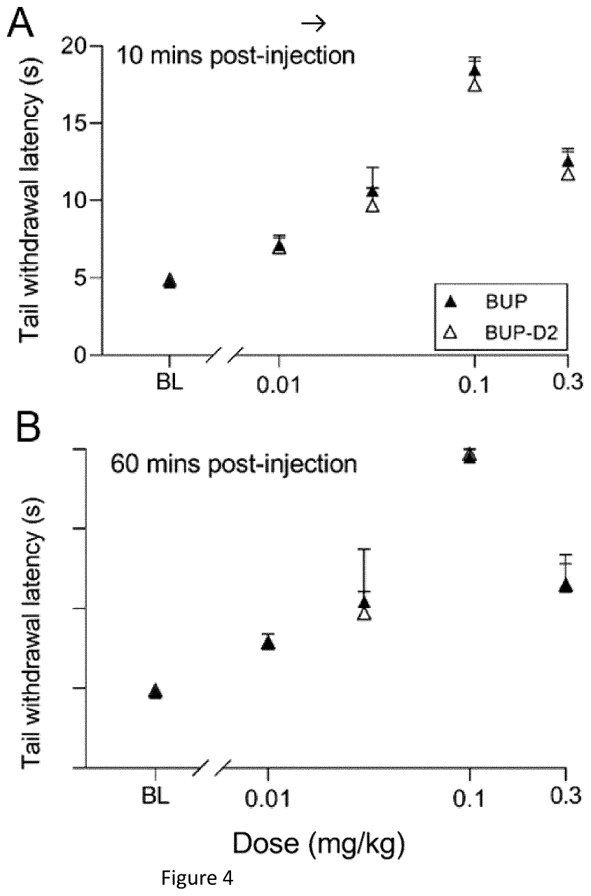

We next tested BUP-D₂ in a rat model of analgesia (called “antinociception” in non-human animals) to determine the potency and efficacy of BUP-D₂ in a whole-organism model of acute opioid exposure. We used the warm water tail withdrawal assay, which measured the latencies of rats to remove their tails from 50° C. water before (baseline) and after (10 and 60 minutes) an intravenous injection of BUP-D₂ or BUP. At baseline, this temperature of water elicits a tail withdrawal response after approximately five seconds (FIG. 4), and administration of an analgesic/antinociceptive agent increases the latency of response. BUP-D₂ and BUP dose-dependently increased tail withdrawal latency 10 and 60 minutes after administration (FIG. 4), up to the investigator-imposed maximum of 20 seconds. At both time points, there were no differences between BUP and BUP-D₂ at any dose in potency and efficacy of tail withdrawal latency (Table 2). This finding suggests that BUP-D₂ can cross the blood-brain barrier and bind opioid receptors in the brains of mammals, providing evidence that BUP-D₂ can likely cross the more permeable placental barrier and block full agonist opioids from binding opioid receptors in the fetal brain.

TABLE 2
Potency and efficacy of BUP and BUP-D₂ antinociception,
mean (95% confidence interval)

| | BUP | BUP-D2 |
|---|---|---|
| 10 minutes | | |
| ED₅₀ (mg/kg) | 0.025 (0.012-0.049) | 0.021 (0.0074-0.049) |
| % MPE (at 0.1 mg/kg) | 90.1 (68.8-111.4) | 84.4 (43.5-125.2) |
| 60 minutes | | |
| ED₅₀ (mg/kg) | 0.021 (0.0043-0.063) | 0.018 (0.0045-0.049) |
| % MPE (at 0.1 mg/kg) | 96.8 (83.2-110.5) | 97.9 (89.1-106.8) |

% MPE, percent maximum possible effect

To summarize, these results indicate that BUP-D₂ exhibits pharmacodynamic properties necessary for blocking fentanyl and other potent full agonists.

### Relative to BUP, BUP-D₂ Resists Metabolism to NorBUP

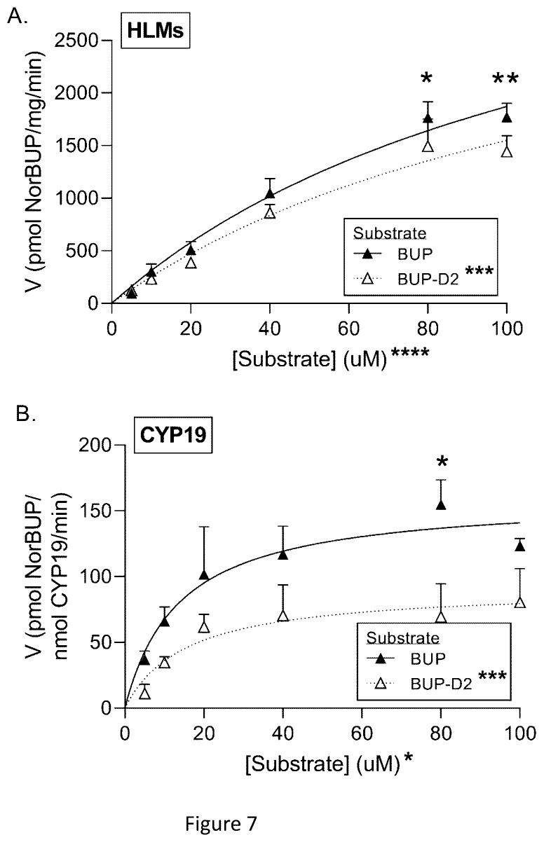

Several types of cytochrome P450 enzymes (CYPs), including CYPs 3A4, 3A5, 2C8, and 19, metabolize BUP to NorBUP (i.e., aromatase) [13]. For this reason, to determine whether deuterating BUP slows its metabolism, we first measured the velocity of NorBUP formation (V_{NorBUP}) from BUP or BUP-D₂ by human liver microsomes (HLMs), which contain the numerous types of CYPs that the human liver expresses. A two-way ANOVA determined there was an interaction effect between substrate type (BUP or BUP-D₂) and substrate concentration, and a Sidek's multiple comparisons test determined that V_{NorBUP} was slower for BUP-D₂ than for BUP at the two highest substrate concentrations (FIG. 7, Panel A). Next, we determined the velocities of BUP and BUP-D₂ metabolism to NorBUP by the major placental enzyme known to metabolize BUP to NorBUP, CYP 19. Using SuperSomes™ transfected with human CYP19, we determined that BUP-D₂ metabolism by CYP19 was appreciably slower than CYP 19 metabolism of BUP (FIG. 7, Panel B). A two-way ANOVA determined there was a main effect of substrate type and substrate concentration, and a Sidek's multiple comparisons test determined that V_{NorBUP} was slower for BUP-D₂ than for BUP at 80 μM substrate concentration. These findings suggest that BUP-D₂ can decrease placental NorBUP concentration and subsequently decrease transfer of NorBUP to the fetus.

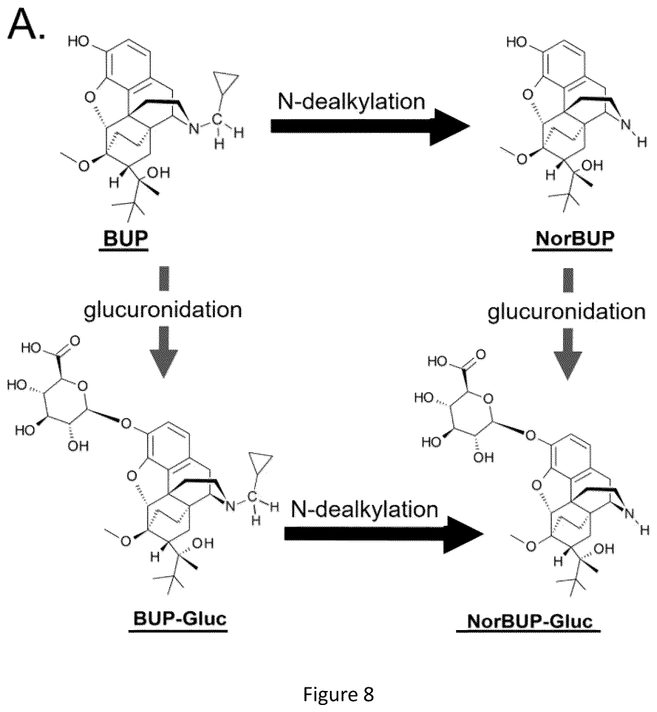

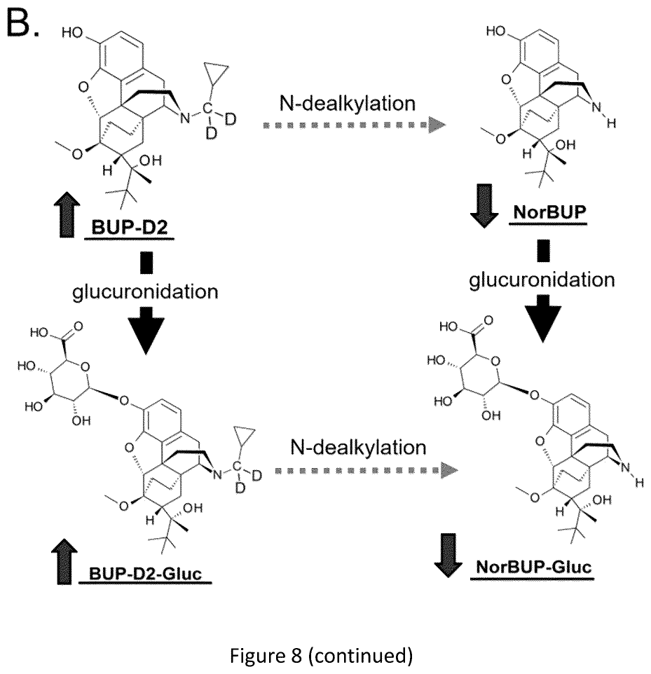

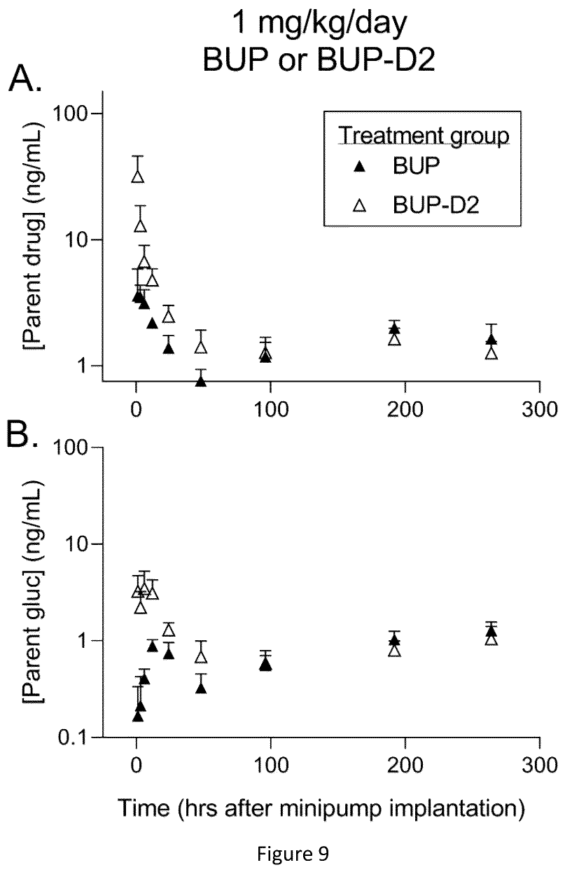

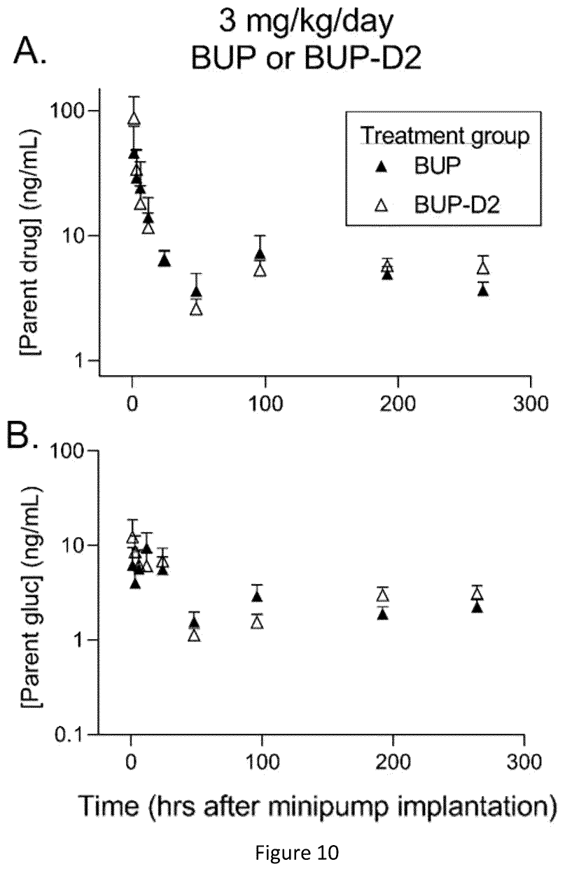

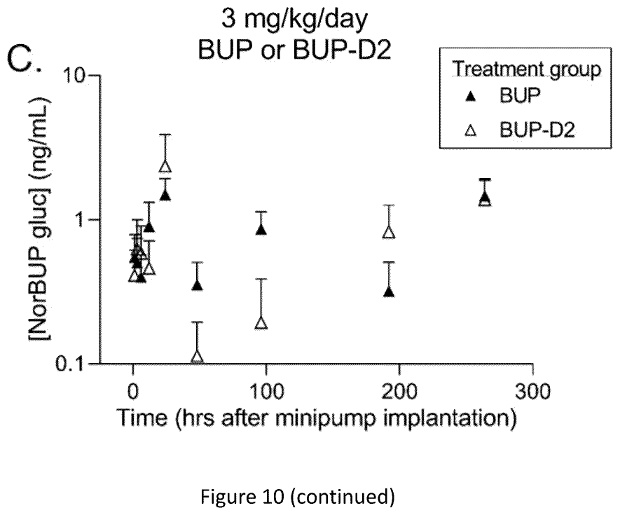

To determine whether fetal brain concentrations of NorBUP were lower following BUP-D₂ administration relative to BUP administration, we dosed pregnant Long Evans rats with BUP or BUP-D₂ (1 or 3 mg/kg/day) via subcutaneously implanted osmotic minipumps from gestation day 9-20. On gestation day 20, which is approximately 2 days before delivery, we harvested fetal brains and measured concentrations of BUP, BUP-D₂, NorBUP, NorBUP glucuronide, BUP glucuronide, and BUP-D₂ glucuronide via LC/MS/MS analysis. We also measured maternal plasma concentrations of these analytes across gestation following minipump implantation. In addition to NorBUP, the glucuronide conjugates are major BUP metabolites in humans and rodents, increased concentrations of glucuronide conjugates may indicate shunting from the NorBUP pathway (FIG. 8). Surprisingly, we detected no NorBUP in fetal brains or maternal plasma, regardless of drug (BUP or BUP-D₂), dose (1 or 3 mg/kg/day, which are considered high doses of BUP), or time point (for maternal plasma). However, we observed greater maximum maternal plasma concentrations (Cₘₐₓ) of BUP-D₂ vs. BUP and of BUP-D₂ glucuronide vs. BUP glucuronide for the 1 mg/kg/day dose (FIG. 9, Panels A and B). Area under the curve for BUP-D₂ was significantly greater than area under the curve for BUP (data not shown). These data provide evidence that BUP-D₂ metabolism is slowed and the glucuronide conjugate pathway is enhanced relative to BUP. This effect was not observed at the higher dose (FIG. 10), possibly due to saturation of metabolic enzymes leading to a ceiling effect on plasma concentrations of the metabolites. There were no group differences in analyte concentrations in the fetal brains (data not shown). A strength of this experiment is that BUP-D₂ was administered chronically throughout gestation, as it would be administered in humans. An obvious weakness is the lack of NorBUP formation in the rat model. This was unexpected because, in pregnant humans taking BUP for opioid use disorder, maternal plasma concentrations of NorBUP are similar to those of BUP [14], suggesting that NorBUP likely has a greater effect on pregnant humans than pregnant rats.

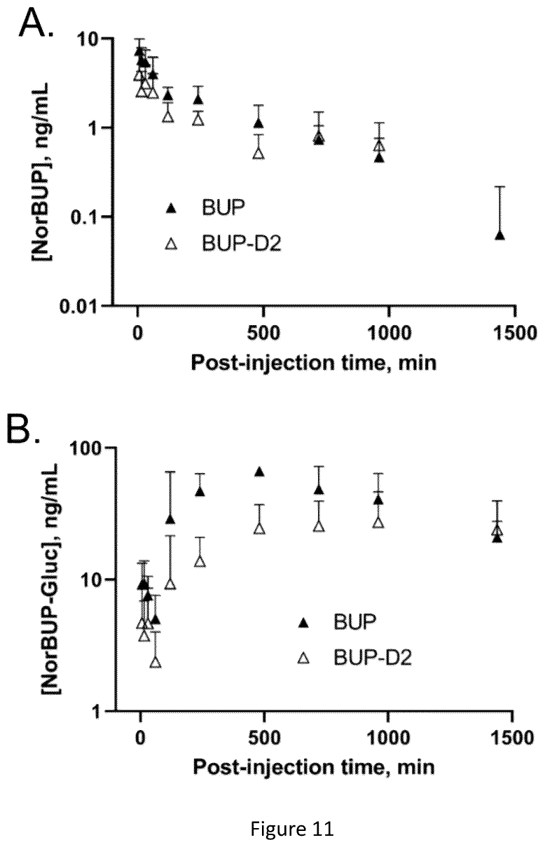

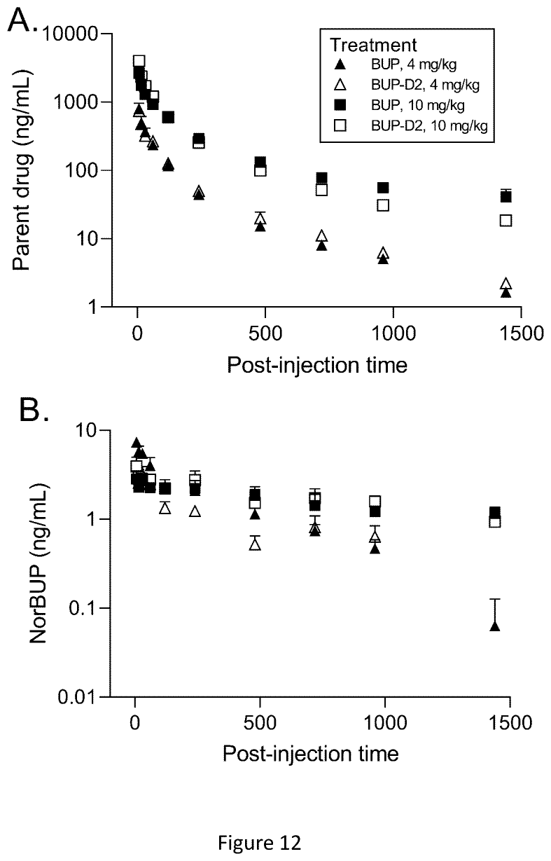

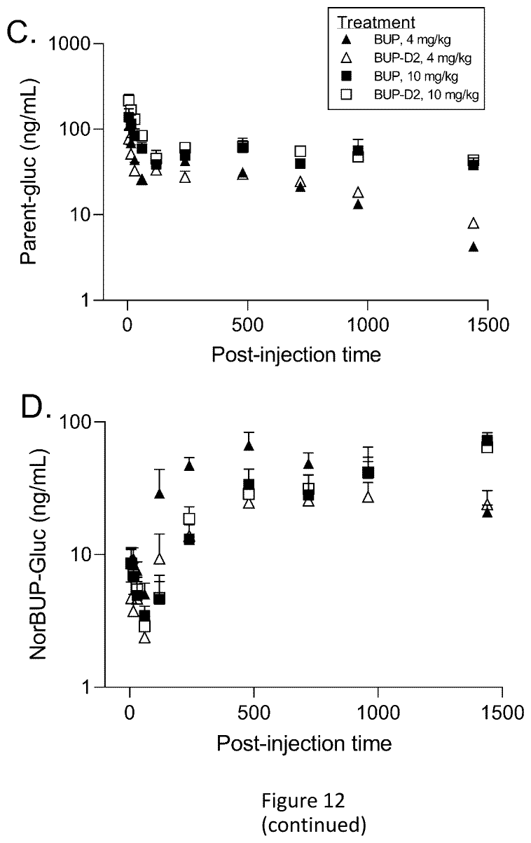

To increase the chances of measuring NorBUP formation (and therefore testing our hypothesis) in a rat model, we replicated a pharmacokinetics study that successfully measured NorBUP following BUP administration [15]. In this study, a single intravenous bolus dose of BUP was administered to male Sprague Dawley rats, and plasma concentrations of BUP and NorBUP were measured at multiple time points, up to 24 hours, after administration [14]. Likewise, we administered BUP or BUP-D₂ to male Sprague Dawley rats and measured plasma concentrations of parent drugs, NorBUP, or their glucuronides. For the smaller dose (4 mg/kg), we observed a small but insignificant decrease in plasma concentrations of NorBUP in the BUP-D₂ group relative to the BUP group (FIG. 11, Panel A). The group difference was much more pronounced for NorBUP glucuronide (FIG. 11, Panel B), a metabolite of NorBUP and BUP (or BUP-D₂) glucuronide (FIG. 8). Area under the NorBUP glucuronide curve was significantly greater for the BUP versus BUP-D₂ group (data not shown). This suggests that a smaller pool of NorBUP is available for glucuronidation following BUP-D₂ administration than following BUP administration. A human study demonstrated that plasma concentrations of NorBUP glucuronide are approximately five-fold greater than NorBUP plasma concentrations following BUP administration [16], while our study in rats showed up to 30-fold more NorBUP glucuronide (FIG. 11, Panels A and B). This suggests there is more extensive glucuronidation of NorBUP in rats relative to humans, and that NorBUP levels in humans would be more greatly altered by precision deuteration than in rats. There were no group differences following the larger dose (10 mg/kg) (FIG. 12, Panels A-D). This is possibly due to saturation of metabolism, as evidenced by increases in plasma concentrations of BUP and BUP-D₂ that are proportional to the increase in dosing (FIG. 12, Panel A), but no concomitant increase in plasma concentrations of metabolites (FIG. 12, Panels B-D).

### BUP-D₂-Mediates Mitigation of Fentanyl-Induced Catalepsy in Pregnant Sprague-Dawley Rats

During gestational days (GD) 8-9, osmotic minipumps administering either a vehicle (1% dimethyl sulfoxide; n=3), 0.1 mg/kg/day BUP (n=4), or 0.1 mg/kg/day BUP-D₂ (n=4) were subcutaneously implanted in pregnant Sprague-Dawley rats. This was accompanied by daily subcutaneous fentanyl (FENT) injections at a dosage of 100 μg/kg from GD 13 to GD 22 across all treatment groups. This preclinical model is designed to parallel the clinical scenario of pregnant women undergoing opioid maintenance therapy who are experiencing a fentanyl relapse during pregnancy. We utilized catalepsy duration (in seconds) with a maximum cutoff of 30 seconds as a proxy marker of opioid toxicity. Catalepsy was measured during early fentanyl relapse (GD 14) and late gestation (GD 20).

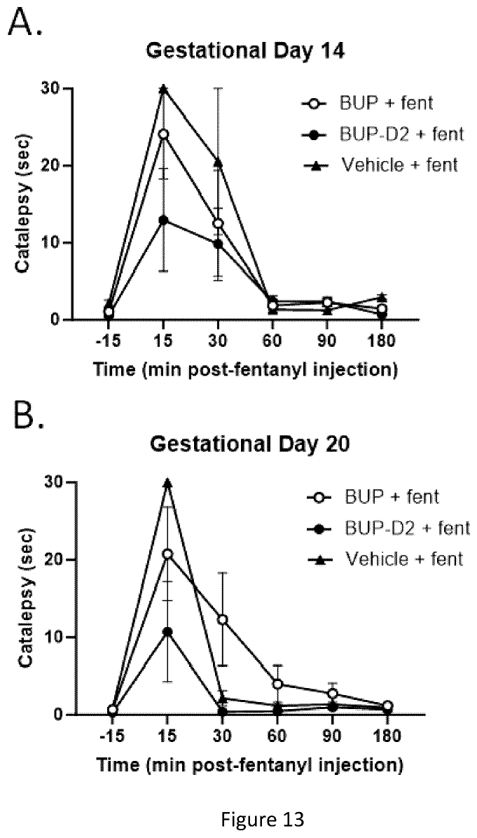

On GD 14, the maximum group mean of catalepsy for dams receiving only FENT was 30 seconds (SEM=0.00; FIG. 13, Panel A). The maximum group mean for dams receiving BUP+FENT was slightly lower (mean=24.13; SEM=5.875). In contrast, the maximum group mean for dams receiving BUP-D₂+FENT was considerably lower (mean=12.95; SEM=6.666). Each group reached its maximum group mean value 15 minutes after FENT injection, and returned to pre-FENT levels 60 minutes after FENT injection.

Results on GD 20 were similar to those on GD 14. On GD 20, the maximum group mean of catalepsy for dams receiving only FENT was 30 seconds (SEM=0.00; FIG. 13, Panel B). The maximum group mean for dams receiving BUP+fentanyl was slightly lower (mean=20.78; SEM=5.997). In contrast, the maximum group mean for dams receiving BUP-D₂+FENT was considerably lower (mean=10.73; SEM=6.450). Each group reached its maximum group mean value 15 minutes after FENT injection. The BUP-D₂+FENT and Vehicle+FENT groups returned to pre-FENT levels 30 minutes after FENT injection, while the BUP+FENT group required over 60 minutes to return to pre-FENT levels.

These results suggest that BUP-D₂ provides enhanced protection against fentanyl-induced catalepsy compared to BUP.

BUP-D₂ Decreases Neonatal Opioid Withdrawal Syndrome (NOWS) in the Presence of Prenatal Fentanyl Exposure More Effectively than BUP

This experiment investigates the relative efficacy of BUP and BUP-D2 in decreasing neonatal opioid withdrawal syndrome (NOWS) in the presence of prenatal fentanyl exposure. Both female and male rat pups born to the Sprague-Dawley rats subjected to the foregoing catalepsy study were evaluated for NOWS within a 4-12 hour post-parturition window. An indicator of withdrawal, distance moved (cm), was quantified using Noldus Ethovision software as we previously described [2, 17]. Two doses of naltrexone (0 and 1 mg/kg) were used to precipitate withdrawal signs.

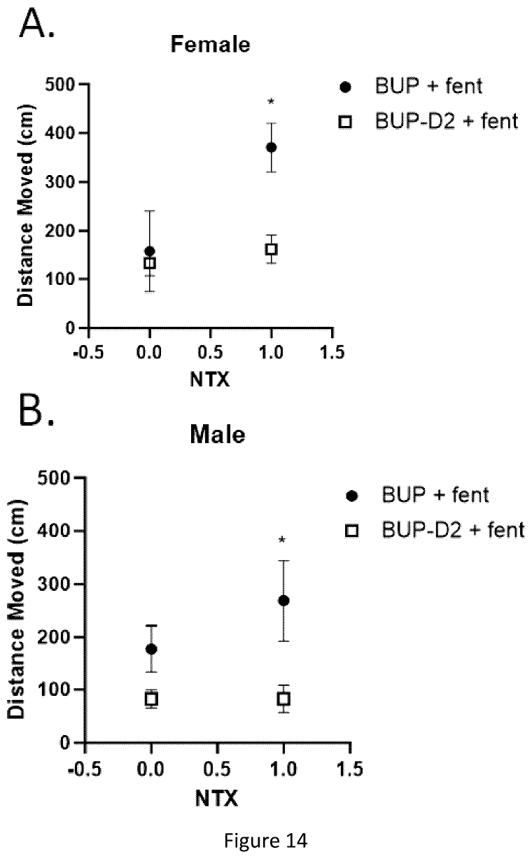

In females (FIG. 14, Panel A) and males (FIG. 14, Panel B), naltrexone significantly precipitated withdrawals in pups that were prenatally exposed to BUP+FENT, but not in pups that were prenatally exposed to BUP-D₂+FENT. A two-way ANOVA was executed, incorporating the factors treatment (BUP+FENT and BUP-D₂+FENT) and naltrexone (NTX; 0 and 1 mg/kg). In female pups, there was a significant treatment effect [F (1, 12)=5.045; *p=0.0443] and NTX effect [F (1, 12)=5.392; *p=0.0386](FIG. 14, Panel A). No significant interaction effect was identified. In male pups, there was a significant treatment effect [F (1, 12)=9.035; *p=0.0109; FIG. 2B] but no naltrexone effect or interaction effect. Within each sex, Šidák's multiple comparisons test indicated that BUP+FENT pups exhibited significantly greater withdrawal than BUP-D2+FENT pups (FIG. 14, Panels A and B).

The data imply that BUP-D₂ has greater efficacy than BUP in mitigating NOWS in the presence of prenatal FENT exposure. This underscores the potential application of BUP-D₂ as a safer therapeutic strategy for the offspring in the management of opioid dependency during pregnancy than BUP.

In summary, BUP-D₂ can protect pregnant people and their fetuses by acting as a high affinity, low efficacy antagonist of full agonist opioids that are often used during relapse. BUP-D₂ has the potential of being an improvement over BUP by resisting metabolism to NorBUP, an active metabolite thought to contribute to NOWS.

### REFERENCES

1. Griffin, B. A., et al., In Utero Exposure to Norbuprenorphine, a Major Metabolite of Buprenorphine, Induces Fetal Opioid Dependence and Leads to Neonatal Opioid Withdrawal Syndrome. J Pharmacol Exp Ther, 2019. 370(1): p. 9-17.
2. Tobacyk, J., Parks, B.J., Salazar, P., Coward, L. U., Berquist, M. D., Gorman, G. S., Brents, L. K., 2023. Interaction between buprenorphine and norbuprenorphine in neonatal opioid withdrawal syndrome. Drug Alcohol Depend 249, 110832.
3. Huang, P., et al., Comparison of pharmacological activities of buprenorphine and norbuprenorphine: norbuprenorphine is a potent opioid agonist. J Pharmacol Exp Ther, 2001. 297(2): p. 688-95.
4. Kacinko, S. L., et al., Correlations of maternal buprenorphine dose, buprenorphine, and metabolite concentrations in meconium with neonatal outcomes. Clin Pharmacol Ther, 2008. 84(5): p. 604-12.
5. Concheiro, M., et al., Maternal buprenorphine dose, placenta buprenorphine, and metabolite concentrations and neonatal outcomes. Ther Drug Monit, 2010. 32(2): p. 206-15.
6. Shah, D., et al., Predictors of neonatal abstinence syndrome in buprenorphine exposed newborn: can cord blood buprenorphine metabolite levels help? Springerplus, 2016. 5(1): p. 854.
7. Uttamsingh, V., et al., Altering metabolic profiles of drugs by precision deuteration: reducing mechanism-based inhibition of CYP2D6 by paroxetine. J Pharmacol Exp Ther, 2015. 354(1): p. 43-54.
8. Harbeson, S. L., et al., Altering Metabolic Profiles of Drugs by Precision Deuteration 2: Discovery of a Deuterated Analog of Ivacaftor with Differentiated Pharmacokinetics for Clinical Development. J Pharmacol Exp Ther, 2017. 362(2): p. 359-367.
9. Miwa, G. T., et al., The use of intramolecular isotope effects to distinguish between deprotonation and hydrogen atom abstraction mechanisms in cytochrome P-450- and peroxidase-catalyzed N-demethylation reactions. J Biol Chem, 1983. 258(23): p. 14445-9.
10. Raynor, K., et al., Pharmacological characterization of the cloned kappa-, delta-, and mu-opioid receptors. Mol Pharmacol, 1994. 45(2): p. 330-4.
11. Toll, L., et al., Standard binding and functional assays related to medications development division testing for potential cocaine and opiate narcotic treatment medications. NIDA Res Monogr, 1998. 178: p. 440-66.
12. Raynor, K., et al., Characterization of the cloned human mu opioid receptor. J Pharmacol Exp Ther, 1995. 272(1): p. 423-8.
13. Picard, N., et al., In vitro metabolism study of buprenorphine: evidence for new metabolic pathways. Drug Metab Dispos, 2005. 33(5): p. 689-95.
14. Concheiro, M., et al., Preliminary buprenorphine sublingual tablet pharmacokinetic data in plasma, oral fluid, and sweat during treatment of opioid-dependent pregnant women. Ther Drug Monit, 2011. 33(5): p. 619-26.
15. Gopal, S., T. B. Tzeng, and A. Cowan, Characterization of the pharmacokinetics of buprenorphine and norbuprenorphine in rats after intravenous bolus administration of buprenorphine. Eur J Pharm Sci, 2002. 15(3): p. 287-93.
16. Moody, D. E., et al., Gender differences in pharmacokinetics of maintenance dosed buprenorphine. Drug Alcohol Depend, 2011. 118(2011):p. 479-83.
17. Kulbeth, H. J., Fukuda, S., Brents, L. K., 2021. Automated quantification of opioid withdrawal in neonatal rat pups using Ethovision® XT software. Neurotoxicol Teratol 84, 106959.

1. A method for fetal protection against full agonist opioid exposure, the method comprising administering a deuterated buprenorphine, or a pharmaceutically acceptable salt thereof, to a maternal subject during the maternal subject's pregnancy or in anticipation of the maternal subject's pregnancy.

2. The method of claim 1, wherein the maternal subject is administered an effective amount of the deuterated buprenorphine to prevent or alleviate fetal opioid dependence and/or neonatal opioid withdrawal syndrome.

3. The method of claim 2, wherein the deuterated buprenorphine is administered chronically during fetal gestation.

4. The method of claim 1, wherein the deuterated buprenorphine comprises a formula of 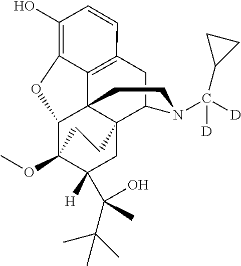
((4aS,6R,7R,7aR,12bS)-3-(cyclopropylmethyl-d2)-6-((S)-2-hydroxy-3,3-dimethylbutan-2-yl)-7-methoxy-1,2,3,4,5,6,7,7a-octahydro-4a,7-ethano-4,12-methanobenzofuro[3,2-e]isoquinolin-9-ol).

5. The method of claim 1, wherein the maternal subject has, or is suspected of having, an opioid addiction, and/or dependence.

6. The method of claim 5, wherein the maternal subject has opioid use disorder.

7. The method of claim 1, wherein the maternal subject is exposed to an opioid during the subject's pregnancy or suffers from opioid withdrawal or pain.

8. (canceled)

9. The method of claim 1, further comprising administering the deuterated buprenorphine to the maternal subject's neonate.

10. (canceled)

11. The method of claim 1, wherein the deuterated buprenorphine has a higher affinity than morphine or fentanyl for human mu opioid receptor (hMOR), human delta opioid receptor (hDOR), human kappa opioid receptor (hKOR), or any combination thereof.

12. (canceled)

13. The method of claim 1, wherein the deuterated buprenorphine has an isotopic enrichment factor of at least 3500.

14. A method for fetal protection, the method comprising administering a deuterated buprenorphine to a fetal subject.

15. The method of claim 14, wherein the fetal subject is administered an effective amount of the deuterated buprenorphine to prevent or alleviate neonatal opioid withdrawal syndrome in the fetal subject.

16. The method of claim 15, wherein the deuterated buprenorphine is administered chronically during fetal gestation.

17. The method of claim 14, wherein the deuterated buprenorphine comprises a formula of 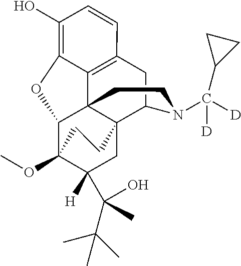
((4aS,6R,7R,7aR,12bS)-3-(cyclopropylmethyl-d2)-6-((S)-2-hydroxy-3,3-dimethylbutan-2-yl)-7-methoxy-1,2,3,4,5,6,7,7a-octahydro-4a,7-ethano-4,12-methanobenzofuro[3,2-e]isoquinolin-9-ol).

18. The method of claim 14, wherein the fetal subject is gestating in a maternal subject having, or suspected of having, an opioid addiction.

19. The method of claim 18, wherein the subject has opioid use disorder.

20. The method of claim 14, wherein the fetal subject is gestating in a maternal subject exposed to an opioid or suffering from opioid withdrawal or pain.

21. (canceled)

22. The method of claim 14 further comprising administering the deuterated buprenorphine to the fetal subject subsequent to birth.

23. (canceled)

24. The method of claim 14, wherein the deuterated buprenorphine has a higher affinity than morphine or fentanyl for human mu opioid receptor (hMOR), human delta opioid receptor (hDOR), human kappa opioid receptor (hKOR), or any combination thereof.

25. (canceled)

26. The method of claim 14, wherein the deuterated buprenorphine has an isotopic enrichment factor of at least 3500.

27. A pharmaceutical composition comprising an effective amount of deuterated buprenorphine and a pharmaceutically acceptable carrier, excipient, or diluent.

28.-32. (canceled)
